(19) United States
(12) Patent Application Publication
(10) Pub. No.: US 2024/0043618 A1
(43) Pub. Date: Feb. 8, 2024

(54) ANTIMICROBIAL COMPOSITIONS

(71) Applicant: University of South Florida, Tampa, FL (US)

(72) Inventors: Ramesh Ayyala, Tampa, FL (US); Nurettin Sahiner, Tampa, FL (US)

(21) Appl. No.: 18/380,965

(22) Filed: Oct. 17, 2023

Related U.S. Application Data
(63) Continuation of application No. PCT/US2022/025367, filed on Apr. 19, 2022.
(60) Provisional application No. 63/176,632, filed on Apr. 19, 2021.

(51) Int. Cl. C08G 73/02; A61P 31/04; A01P 1/00

(52) U.S. Cl. CPC C08G 73/0273; C08G 73/0246; A61P 31/04; A01P 1/00

(57) ABSTRACT

This disclosure relates to antimicrobial compositions, and more particularly to treatment of surfaces (e.g., face masks) for the reduction or prevention of transmission of microbes (e.g., bacteria, fungus, and/or viruses). In some embodiments, the compositions comprise a cationic polymer selected from the group consisting of: a polydiallyldimethylammonium salt, a polyethyleneimine salt, a polydiallyldimethylammonium with different counter ions, a chemically modified polyethyleneimine salt, or a combination thereof.

### CROSS REFERENCE TO RELATED APPLICATIONS

This application is a continuation of and claims priority to PCT International Application No. PCT/US2022/025367, entitled “Antimicrobial Compositions”, filed Apr. 19, 2022, which is a nonprovisional of and claims priority to U.S. Provisional Patent Application Ser. No. 63/176,632, entitled “Antimicrobial Compositions”, filed Apr. 19, 2021, the contents of each of which are hereby incorporated by reference into this disclosure.

### TECHNICAL FIELD

This disclosure relates to antimicrobial compositions, and more particularly to treatment of surfaces (e.g., face masks) for the reduction or prevention of transmission of microbes (e.g., bacteria, fungus, and/or viruses).

### BACKGROUND OF THE INVENTION

The COVID-19 pandemic has brought into sharp focus the need for a safe and effective face mask that protects the wearer and is also inexpensively and freely available to the public. N95 and surgical masks need specialized manufacturing, are often in short supply and can be beyond the means of many people in the world. The purpose of general public masking is to decrease transmission from infected asymptomatic carriers to susceptible persons in close vicinity. A technology that can transform any mask, such as a homemade cotton face mask, to a face mask that can prevent transmission of infection and protect the wearer by trapping and killing bacteria, fungi, and/or viruses on contact would be useful.

### SUMMARY OF THE INVENTION

There is a need for personal protection equipment (PPE), especially surgical masks, for people of all ages to prevent the spread and contraction of transmissible disease. Pandemics, such as the Corona virus (COVID-19) pandemic, illustrate the need for easy to use, simple, reliable, effective, and reusable mask options. Masks that are capable of destroying airborne pathogens, thus preventing the secondary risk of infections and contaminations, would be useful. Respirator style masks, such as N95 masks, can be problematic because they are difficult to wear and maintain; they have a limited time usage (about 2 hours); they are expensive; breathing can be difficult; and they can include special requirements for disposal as a biohazard. For these reasons, N95 masks can be impractical for widespread use by large populations of people.

On the other hand, surgical masks can effectively be used to prevent the spread of airborne pathogenic aerosols, provided that they are readily available and can inherently eliminate the accumulated pathogens. In addition to the pathogenic aerosol properties (size of the pathogen, pathogen density in the aerosol, and velocity), the mask filter characteristics such as a diameter of the fiber, thickness, packing density, its construct (e.g., woven or nonwoven), the surface properties of the fibers (e.g., smooth or rough), and the functional groups (e.g., charge), are parameters that determine the effectiveness of the surgical masks. One concern is the deactivation of the accumulated pathogens to prevent transmission and contamination from the virus loaded fibers of the mask during usage and disposal. Re-sterilization of a mask without damaging mask filter characteristics is unlikely. Attempts have been made to modify the fibers to render the mask with antibacterial properties such as treating the material of the mask with iodine and metal. See, Lee, J. H. et al., Efficacy of Iodine-Treated Biocidal Filter Media Against Bacterial Spore Aerosols. J. Appl. Microbiol. 105, 1318-1326 (2008); Zhao, N. et al., Thermoplastic Semi-IPN of Polypropylene (PP) and Polymeric N-halamine for Efficient and Durable Antibacterial Activity. Eur. Polym. J. 47, 1654-1663 (2011); Cerkez, I., et al., Antimicrobial Surface Coatings for Polypropylene Nonwoven Fabrics. React. Funct. Polym. 73, 1412-1419 (2013); Davison, A. M. Pathogen Inactivation and Filtration Efficacy of a New Anti-Microbial and Anti-Viral Surgical Facemask and N95 Against Dentistry-Associated Microorganisms. International dentistry Australasian edition 7, 36-42 (2012); Borkow, G., et al., A Novel Anti-Influenza Copper Oxide Containing Respiratory Face Mask. PLoS One 5, e 11295 (2010); and Li, Y., Leung, et al., Antimicrobial Effect of Surgical Masks Coated with Nanoparticles. J. Hosp. Infect. 62, 58-63 (2006). In some examples, attempts have been made to modify the fibers to render the mask with antibacterial properties such as by functionalization of the main fibers of the surgical mask with sodium chloride salt.

In some embodiments, disclosed herein is a composition comprising a cationic polymer selected from the group consisting of: a polydiallyldimethylammonium salt, a branched polyethyleneimine salt, a chemically modified polydiallyldimethylammonium salt, a chemically modified polyethyleneimine salt, and combinations thereof.

In some embodiments, the composition further comprises a carrier. In some embodiments, the carrier comprises water, alcohol, glycerol, polyethylene glycol, and combinations thereof. In some embodiments, the alcohol is selected from the group consisting of ethanol, propanol, and combinations thereof. In some embodiments, the carrier comprises water. In some embodiments, the salt is a halide salt, an acetate salt, a citrate salt, a borate salt, a phosphate salt, and combinations thereof. In some embodiments, the salt is a chloride salt, a bromide salt, or an iodide salt. In some embodiments, the salt is an acetate salt, a citrate salt, a borate salt, or a phosphate salt. In some embodiments, the chemically modified polydiallyldimethylammonium salt is a polydiallyldimethylammonium salt with a different counter ion. In some embodiments, the chemically modified polyethyleneimine salt is a betainized polyethyleneimine salt. In some embodiments, the chemically modified polyethyleneimine salt is a sulfobetainized polyethyleneimine salt. In some embodiments, the chemically modified polyethyleneimine salt is a phosphobetainized polyethyleneimine salt. In some embodiments, the composition comprises from about 0.5% to about 5% by weight (i.e., w/w) of the cationic polymer.

In some embodiments, the composition can be used as a broad-spectrum antimicrobial agent to treat infections in animals. In some embodiments, the composition is a solution to treat eye infections in animals. In some embodiments, the solution is applied as a gel or ointment to skin infections. In some embodiments, the solution is applied prior to surgery and in the postoperative period as a gel or ointment to be applied as a prophylaxis against broad spectrum antimicrobial infection.

In some embodiments, disclosed herein is a process for preparing an antibacterial, antifungal, and/or antiviral surface on a material, and the method comprising treatment of at least one surface of the material with an effective amount of any of the compositions described herein. In some embodiments, the material is a PPE mask. In some embodiments, the PPE mask is a cotton mask. In some embodiments, the PPE mask is a surgical mask. In some embodiments, the material is a fabric material. In some embodiments, the fabric material is cotton, linen, silk, wool, and combinations thereof. In some embodiments, the material is clothing. In some embodiments, the material is a hard surface. In some embodiments, the hard surface comprises steel, granite, quartz, plastic, concrete, and combinations thereof.

In some embodiments described herein are methods of reducing exposure of a subject to a bacterial, fungal, and/or viral infection, the method comprising applying an effective amount of a composition as described herein to at least one surface of a material worn by the subject.

In some embodiments, the material is a PPE mask. In some embodiments, the PPE mask is a cotton mask. In some embodiments, the PPE mask is a surgical mask. In some embodiments, the material is a fabric material. In some embodiments, the fabric material is cotton, linen, silk, wool, and combinations thereof. In some embodiments, the material is clothing. In some embodiments, the material is worn on the face of the subject. In some embodiments, the material covers the mouth and/or at least a portion of the nose of the subject. In some embodiments, described herein are face masks comprising a fastening member for attaching a body portion of the face mask to a user; and the body portion joined to the fastening member and configured to be placed over a mouth and at least part of a nose of the user such that inhaled air is drawn through the body portion, the body portion comprising a plurality of layers with at least an outermost layer treated with any of the compositions described herein. In some embodiments, at least the body portion of the face mask is treated with the composition by dipping and squeezing, spraying, ink jet printing, and combinations thereof. In some embodiments, the mask exhibits a reduction in contact transfer of an inoculum of a bacterium, fungus, and/or virus following contact with the mask.

In some embodiments, described herein are face masks, comprising: a body portion configured to be placed over a mouth and at least part of a nose of a user such that inhaled air is drawn through said body portion, wherein said body portion comprises an outer layer treated with an effective amount of any of the compositions described herein. In some embodiments, the body portion comprises a plurality of layers, at least one of which is treated with the composition in an amount effective to deactivate a bacterium, fungus, and/or virus. In some embodiments, at least one layer of the face mask is treated with the composition by a method selected from the group consisting of dipping and squeezing, spraying, ink jet printing, brushing, soaking, and combinations thereof. In some embodiments, the composition is present in an amount of at least about 0.01% to about 20% by weight of the outer layer.

In some embodiments described herein are methods for deactivating a bacterium, fungus, and/or virus on a surface, the method comprising treating the surface with an effective amount of any composition described herein. In some embodiments, the surface is the surface of a fabric material. In some embodiments, the fabric material comprises cotton, linen, silk, wool, and combinations thereof. In some embodiments, the surface is the surface of a face mask. In some embodiments, the composition is present in an amount of at least about 0.01% to about 20% by weight on the surface.

In some embodiments described herein are methods of treating a bacterial infection in a subject in need thereof, comprising administering a therapeutically effective amount of a polydiallyldimethylammonium salt, a branched polyethyleneimine salt, a chemically modified polydiallyldimethylammonium salt, a chemically modified polyethyleneimine salt, and combinations thereof.

In some embodiments, the bacterial infection is caused by a bacterium selected from the genus Escherichia, Bacillus, Staphylococcus, Pseudomonas, and combinations thereof. In some embodiments, the bacterial infection is caused by a bacterium selected from the group consisting of E. coli, S. aureus, P. aeruginosa, and B. subtilis, and combinations thereof. In some embodiments, the bacterium that is causing the bacterial infection is gram positive. In some embodiments, the bacterium that is causing the bacterial infection is gram negative.

In some embodiments described herein are methods of treating a viral infection in a subject in need thereof, comprising administering a therapeutically effective amount of a polydiallyldimethylammonium salt, a branched polyethyleneimine salt, a chemically modified polydiallyldimethylammonium salt, a chemically modified polyethyleneimine salt, and combinations thereof. In some embodiments, the virus that causes the viral infection is SARS-CoV-2.

In some embodiments, described herein are methods of treating a fungal infection in a subject in need thereof, comprising administering a therapeutically effective amount of a polydiallyldimethylammonium salt, a branched polyethyleneimine salt, a chemically modified polydiallyldimethylammonium salt, a chemically modified polyethyleneimine salt, and combinations thereof.

In some embodiments, the fungus that causes the fungal infection is from the Candida genus. In some embodiments, the fungus that causes the fungal infection is Candidia albicans. In some embodiments, the method further comprises administering a pharmaceutical acceptable carrier. In some embodiments, the pharmaceutical acceptable carrier is selected from the group consisting of water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and combinations thereof.

### DESCRIPTION OF DRAWINGS

FIG. 1 shows the polymer structures polyethyleneimine (PEI) and Polydiallyldimethylammonium chloride (PDADMACl).

FIG. 2A shows an example of an anion exchange of PEI and acetic acid.

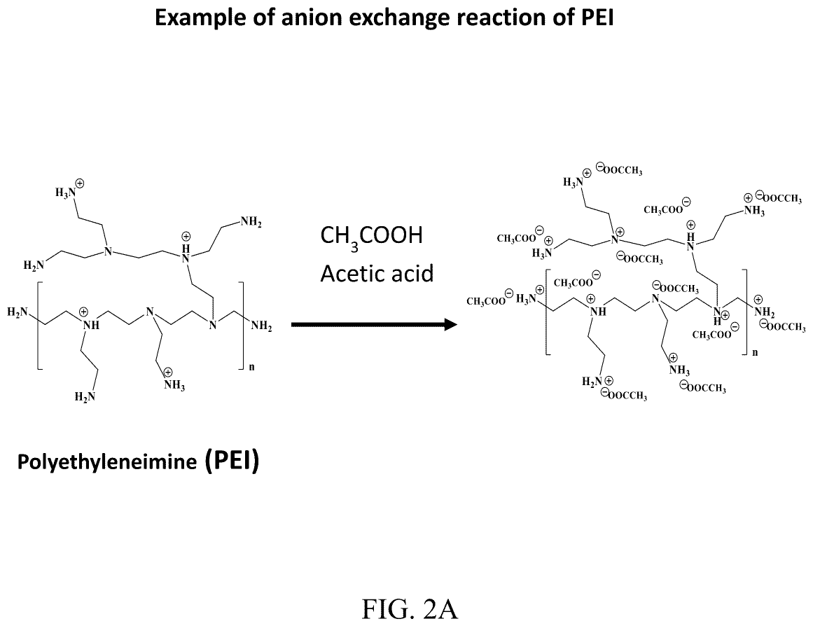

FIG. 2B shows an example of an anion exchange of PEI and citric acid.

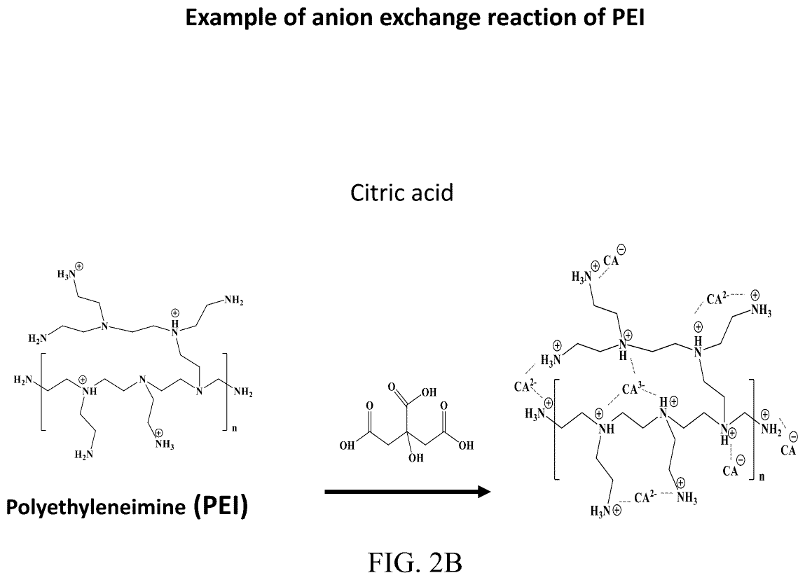

FIG. 3A shows an example of an anion exchange of PDADMACl and acetic acid.

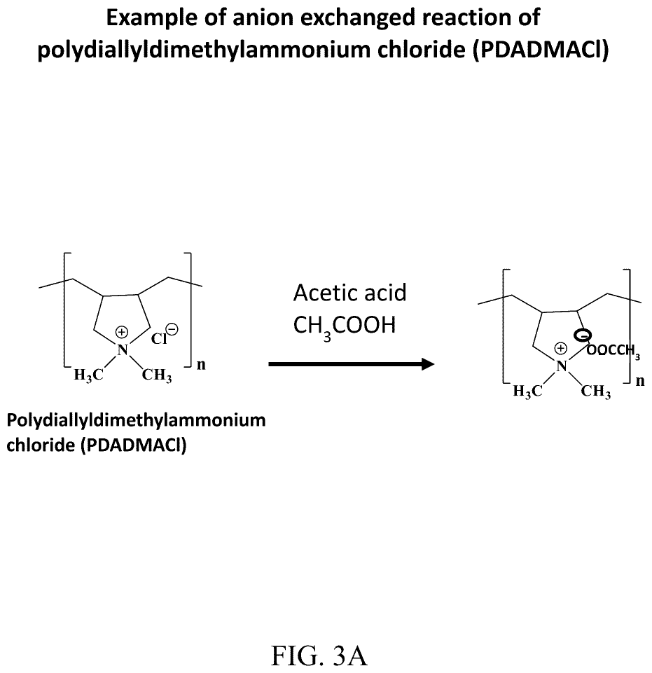

FIG. 3B shows an example of an anion exchange of PDADMACl and citric acid.

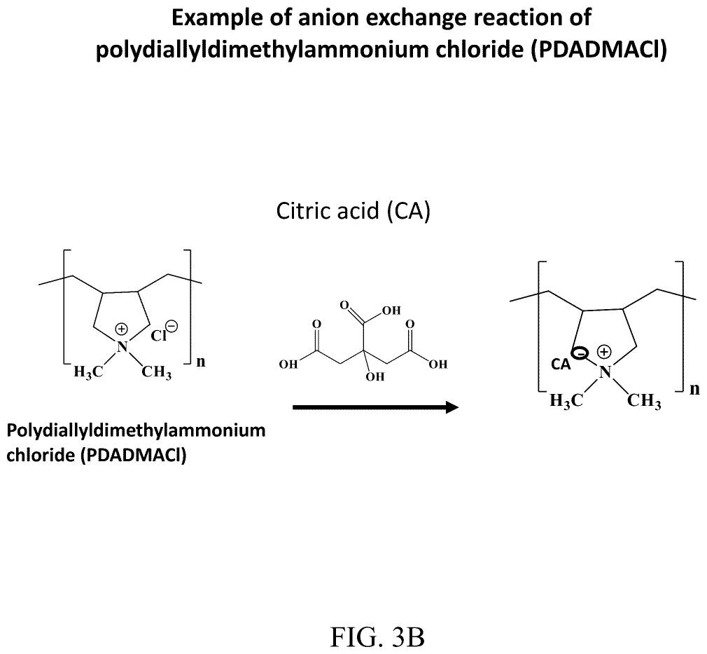

FIG. 4 shows an exemplary scheme for the betainization reaction of polyethyleneimine (PEI) to obtain B-PEI.

FIG. 5 shows spraying of mask cloth with antipathogenic polymer solution.

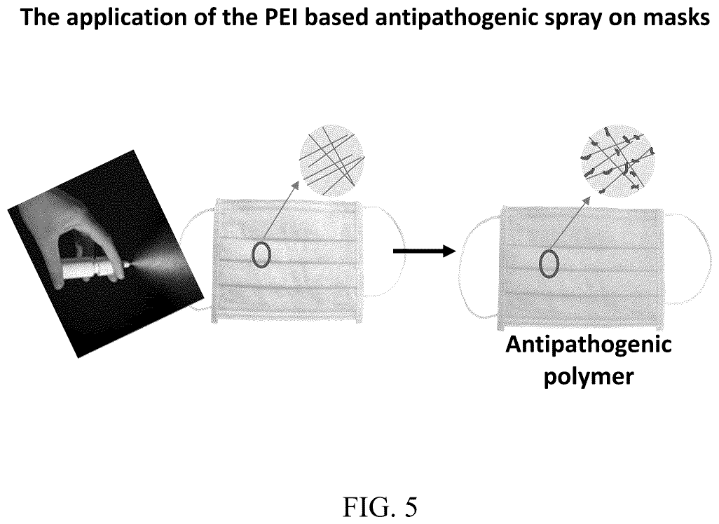

FIG. 6 depicts an example of a face mask.

FIG. 7 is a graph showing the cytotoxicity of L929 fibroblasts in the presence of polyethyleneimine (PEI), polyethyleneimine-citric acid (PEI-CA), polyethyleneimine-boric acid (PEI-BA), polyethyleneimine-hydrogen chloride (PEI-HCl) solutions for 24 h incubation time.

FIG. 8 is a graph showing the cytotoxicity of L929 fibroblasts in the presence of betainized polyethyleneimine (B-PEI), betainized polyethyleneimine-citric acid (B-PEI-CA), betainized polyethyleneimine-boric acid (B-PEI-B A), betainized polyethyleneimine-hydrogen chloride (B-PEI-HCl) solutions for 24 h incubation time.

### DETAILED DESCRIPTION OF THE INVENTION

Infection and disease can occur from contraction of microorganisms such as bacteria, fungi, and viruses which can cause significant mortality and morbidity. The effect of morbidity and mortality is significantly increased if the infection causing pathogen is airborne. To prevent the spread of infections, precautions such as social distancing can be implemented with limited effect. For example, some parts of daily life such as shopping for basic needs (food etc.), postal service, agricultural work, industrial and other related services, and health service necessities include unavoidable contact. Therefore, PPE such as basic face masks to protect against airborne pathogens have a significant role in the prevention of disease transmission. Although current masks can be temporarily effective by preventing the spread of pathogens, the mask itself can quickly become a source of pathogens and require special attention for disposal. The creation of a spray solution with antipathogen properties for application to a mask, can protect the individual's life but also prevent the spread of viruses and eliminate the problems associated with discarding the mask after use. The cloth fabric is generally made up of cellulose fibers that are commonly employed as clothing that can be readily used as face masks. PPEs can turn into effective pathogen fighting tools upon spaying them with an effective antipathogenic polymer solution (antibacterial, antifungal, and antiviral).

There are different categories of face masks. For example, cloth face masks, surgical face masks, and respirator type face masks (e.g., N95 face masks). Apart from the costs involved (cloth masks being the least expensive and N95 being the most expensive), the difference between these example categories is the pore size. Cloth masks are common, particularly in the developing world because they are inexpensive, readily available, and washable. They usually consist of a synthetic or natural cloth material worn across the mouth and nose and with straps, which can be worn behind the head or over the ears to maintain a fit to the face. Surgical masks can be loose-fitting, disposable devices that create a physical barrier between the mouth and nose of the wearer and potential contaminants in the immediate environment. Surgical masks can be made in various thicknesses and with different abilities to protect you from contact with liquids. If worn properly, surgical masks may offer effective blocking against splashes and large-particle droplets, but not against very small particles in the air that may be transmitted by coughs, sneezes, or certain medical procedures.

A respirator type mask, such as a N95 respirator, is a respiratory protective device designed to achieve a close facial fit and very efficient filtration of airborne particles. The ‘N95’ designation means that when subjected to careful testing, the respirator blocks at least 95 percent of very small (0.3 micron) test particles. If properly fitted, the filtration capabilities of N95 respirators exceed those of other face masks. However, even a properly fitted N95 respirator does not completely eliminate the risk of illness or death.

In studies comparing the performance of surgical mask filters using a standardized airflow, filter performance has been shown to be highly variable. Collection efficiency of surgical mask filters can range from less than 10% to nearly 90% for different manufacturers' masks when measured using the test parameters for National Institute for Occupational Safety and Health (NIOSH) certification. The mechanism of action of the filters is based on inertial impaction (stops large particles), interception (by the fibers), diffusion (helps trap small particles), and finally electrostatic attraction (opposite charged particles are attracted to a charged fiber).

Ideally, a mask should be inexpensive, easy to make, and have pore size that can trap 0.3-micron particles >95% of the time and kill the virus or bacteria on contact if possible. Quan et al. Universal and Reusable Virus Deactivation System for Respiratory Protection. Scientific Reports, 2017; 7: 39956 developed a universal, reusable virus deactivation system by functionalization of the main fibrous filtration unit of surgical mask with sodium chloride salt. The salt coating on the fiber surface dissolves upon exposure to virus aerosols and recrystallizes during drying, destroying the pathogens. However, this technique still required the manufacturing of a mask with the salt coated fibers to be in the middle layer.

Kabindra M. et al., Evaluating the Efficacy of Cloth Facemasks in Reducing Particulate Matter Exposure. Journal of Exposure Science and Environmental Epidemiology (2017) 27, 352-357 reported that particulate matter <2.5 micrometer is more harmful than larger particles due to their smaller size as they can readily penetrate human bronchi and lungs. The size of the commonly known microbes e.g., yeast (fungi ˜10 μm), bacteria (E.coli ˜2 μm; Staphylococcus ˜1 μm) and viruses (Covid-19 ˜100 nm, Smallpox ˜300 nm, Rabies ˜150 nm; Influenza-100 nm, Polio & Rinovirus-30 nm) that cause many diseases are in size range of few micrometer to and few tens nanometer. Lydia-Ann J. et al., Nano-Sized and Filterable Bacteria and Archaea: Biodiversity and Function, Frontiers in Microbiology, 2018, 9, article 1971, reported nanosized microorganism presumably in 50-400 nm size range can pass through small pore membranes with pore size 0.22-0.45 micrometer due to absence of their rigid cell wall, that are generally protein decorated lipid membrane. The lack of rigid cell membrane allows nanosized microorganisms (e.g., various viruses) to effectively squeeze through the pores of filters or masks.

Montserrat Bárcena et al., Cryo-electron Tomography of Mouse Hepatitis Virus: Insights into the Structure of the Coronavirion, PNAS, 2009;106(2) 582-587 reported that Coronaviruses (CoVs) are enveloped and plus-stranded RNA viruses capable of infection birds and human have variable sizes and various morphology that are roughly spherical with 80-120 nm size range. The smaller the particles in size of the microbes, the longer the time for particles or microorganisms to stay in air suspended from few hours to few days when they are aerosolized into air by coughing or sneezing.

The rapid spread outbreak of COVID-19 is believed to be facilitated with aerosolized pathogen causing a fast effective infection and transmission. For these reasons, it is important to have uncomplicated and effective protection methods.

Described herein are polymeric aqueous solutions of Polyethyleneimine (PEI) and Polydiallyldimethylammonium chloride (PDADMACl) with various counter ions such as borates, citrates and acetates that can be prepared and used as sprayable solution on a mask that is made of up cloth material such as cellulose and cellulose derivatives and other synthetic polymeric materials. PEI is an antibacterial material. (See Sahin Demirci, et al., PEI-based Ionic Liquid Colloids for Versatile Use: Biomedical and environmental applications, Journal of Molecular Liquids, 2014, 194, 85-92; and Nurettin Sahiner et al., The Synthesis of Desired Functional Groups on PEI Microgel Particles for Biomedical and Environmental Applications. Applied Surface Science, 2015, 354, 380-387).

PEI also comprises characteristics as efficient DNA transfecting materials. (See Liu, Z., et al., Prog. Polym. Sci. 2010, 35, 1144; Vinogradov, S. V. et al., J. Controlled Release 2005, 107, 143; and Xia, T. et al., Nel, A. E. ACS Nano 2009, 3, 3273). PEI also possesses high DNA complexing materials. (See Schafer, J. et al., Biomaterials 2010, 31, 6892; and Boussif, O. et al., Proc. Natl Acad. Sci. USA 1995, 92, 7297). Because of its highly positively charged nature, PEI has a relatively high and efficient capacity to complex with negatively charged DNA. (See Dey, D. et al., Biomaterials 2011, 31, 4647).

PDADMACl is also comprised of positively charged polymeric materials with quaternary ammonium moiety and is well known in its antibacterial properties. (See, Denise Freitas, et al., The Antimicrobial Activity of Free and Immobilized poly(diallyldimethylanunonium) chloride in Nanoparticles of poly(methylmethacrylate), J. Nanobiotechnol, 2015, 13, 58, 2-13; and Carmona-Ribeiro A M, et al., Cationic Antimicrobial Polymers and their Assemblies. Int J Mol Sci, 2013;145):9906-46). Quaternary amine salt containing a polymer with various formulations was successfully tested against various microorganisms. (See Melo L D, et al., Antimicrobial Particles from Cationic Lipid and Polyelectrolytes. Langmuir. 2010;26(14) Carmona-Ribeiro A M, et al., Fungicidal Assemblies and their Mode of Action. OA Biotechnol. 201:3;2:25; and Xue Y, et al., Antimicrobial Polymeric Materials with Quaternary Ammonium and Phosphonium Salts. Int J Mol Sci. 2015;16(2):3626-55).

Ganewatta M S, et al., Controlling Macromolecular Structures Towards Effective Antimicrobial Polymers. Polymer. 2015;63:A1-29; and Taresco V, et al., Antimicrobial and Antioxidant Amphiphilic Random Copolymers to address medical device-centered infections. Acta Biomater. 2015;22:131-40 suggest that cationic polymer (polycations) are polyelectrolytes and generally present high activity because of their highly localized positive charges that foster electrostatic interaction with negatively charged microorganisms or bacterial cell membranes thus leading to the disruption of the cell membrane and/or wall,

Non-limiting examples of cationic polymers or polyelectrolytes with various counter ions include: chloride (Cl—), borates (BO₃⁻, RBO₃²⁻, R₂BO₃¹⁻, R₃BO₃ where R can be organic cations or Na⁻, K⁺), citrates (C₆H₅O₃³⁻, RC₆H₅O₃²⁻, R₂C₆H₅O₃¹⁻, and R₃C₆H₅O₃ where R can be organic cations or Na⁺, K⁺), and acetates (CH₃COO⁻, CH₃COOR where R can be organic cations or Na⁺, K⁺) that can readily impart antibacterial antiviral to the surfaces.

The enhancement of antibacterial and antiviral efficacy of the mask involves spraying, brushing, dipping and or soaking into the corresponding polymer solutions.

Although PEI is a known branched cationic polymer noted for its' highly effective gene delivery capacity and antibacterial properties, the toxic nature of PEI has restricted its widespread use in biomedical applications. However, there is no report on antibacterial or antipathogenic properties of PEI with different cations such as borates, citrates. and acetate. On the other hand, chemical modification, e.g., betainization of PEI make this material biocompatible. (See, Nurettin Sahiner, et al., Can PEI Microgels Become Biocompatible upon Betainization? Materials Science and Engineering: C, 2017;77, 642-648).

Provided herein is a sprayable solution, made of positively charged polymer with different counterions, that when sprayed on any material can create a layer with antipathogenic property with the ability to capture and prevent tiny particles from passing through and eventually deactivate the contacted pathogens that could be virus, bacteria and fungus that are trapped. For example, provided herein are sprayable solutions comprising one or more positively charged polymers. The positively charged polymers can be with different counterions. When sprayed on a material, such solutions can be useful, for example, for preventing pathogens from passing through the material and/or deactivating such pathogens on the material. Surface charges exhibited by bacteria and viruses can be utilized to trap microorganisms on a suitably charged fibers of mask. Disclosed herein is the creation of a positively charged biocompatible polymeric material PEI with antimicrobial properties.

Also provided herein, is an efficient method to transform a cloth or surgical mask into an effective antipathogen barrier as a reusable system via functionalization of fibers of cloth/surgical mask by antipathogenic modification and coatings (e.g., active polymeric solution). Moreover, it is anticipated that by spaying active polymeric solution onto regular cloth/surgical masks, the risk of primary and secondary infection and transmission can be prevented.

The polymeric solution described herein includes an aqueous solution of polymeric materials that can be sprayed onto face masks, clothing, and hard surfaces to deactivate pathogens such as bacteria, fungi, and viruses. Such polymeric materials can include cationic polymer (e.g., the cationic polymers described herein). In some embodiments, the polymeric materials that can be used include PEI and PDADMACl and their chemically modified, and anion exchanged forms.

The chemical modification PEI includes sulfo betainization and phosphate betainization employing various chemical modification agents such as 1,3-propanesultone, 3-chloropropane sulfonic acid, 3-bromopropanesulfonic acid, and 3-(bromopropyl)phosphonic acid. In general, all the chemical modification agents that possess reactive halide in one end of hydrocarbon chain and sulfate or phosphoric groups in the other end of hydrocarbon chain may be used. The number of carbon atoms in hydrocarbon chain can include about C2-C18. Also, alkyl halides can be used in chemical modification of PEI. In some embodiments, the hydrocarbon can be a C2 hydrocarbon to about a C18 hydrocarbon. In some embodiments, the hydrocarbon is a C2 hydrocarbon, C4 hydrocarbon, C6 hydrocarbon, C8 hydrocarbon, C10 hydrocarbon, C12 hydrocarbon, C14 hydrocarbon, C16 hydrocarbon, C18 hydrocarbon, and combinations thereof.

The anion exchanged forms include the anion exchange of positively charged PEI and PDADMACl polymers using aqueous solutions of 1) acetic acid 2) citric acid 3) boric acid and/or 4) phosphoric acid. Also described herein is the treatment of chemically modified PEI with the acid solutions of 1) acetic acid 2) citric acid 3) boric acid and/or 4) phosphoric acid. In some embodiments, the aqueous solution of PEI, PDADMACl and sulfo betainized and phosphate betainized PEI and anion exchanged PEI and PDADMACl in the range of 0.5-5.0% by weight (i.e., w/w) are used as a spray solution. For example, an aqueous solution of about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 4.5% to about 5%, about 4% to about 5%, about 3.5% to about 5%, about 3% to about 5%, about 2.5% to about 5%, about 2% to about 5%, about 1.5% to about 5%, or about 1.5% to about 5% by weight of one or more of: PEI, PDADMACl sulfo-betainized PEI, phosphate-betainized PEI, anion exchanged PEI, and anion exchanged PDADMACl is used as a spray solution.

Other agents can be included in the spray solution such as ethanol, propanol, glycerol, and polyethylene glycol in various concentrations.

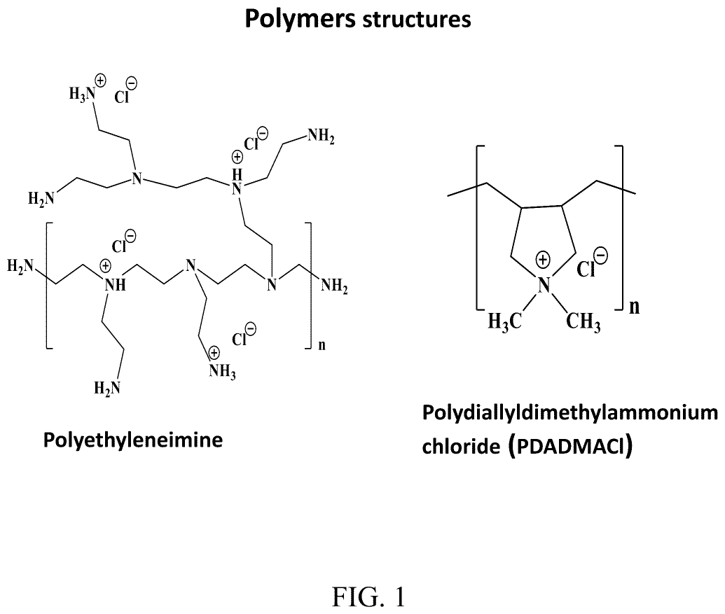

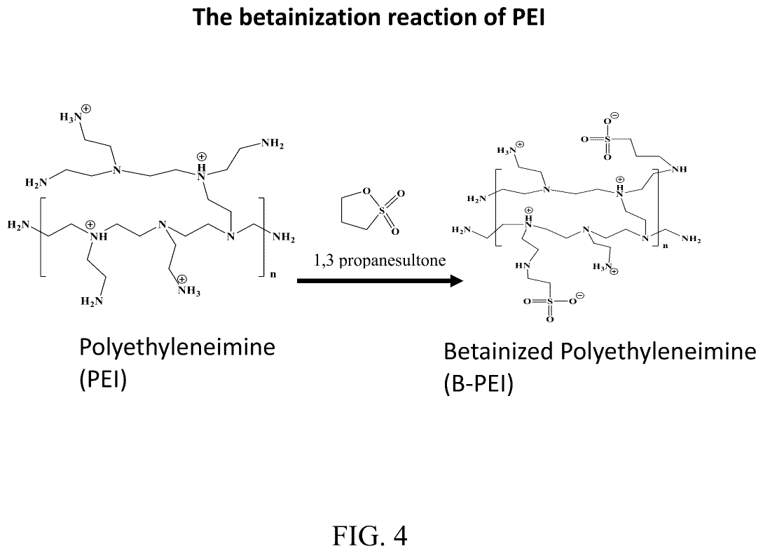

FIG. 4 provides an exemplary scheme for the betainization reaction of PEI to obtain B-PEI. The polymeric materials with the chemical structure given in FIG. 1 are described herein in preparation of solutions (e.g., sprayable solutions) with different counter ions. Non-limiting examples of ionic species that include counter ions are HCL, acetic acid, citric acid, and boric acid. FIG. 1 shows that all of the polymers have a Cl⁻ counter ion, and this counter ion will be replaced by using a different bulkier (e.g., bigger) counter ion such as borate, citrate, and acetate by using their corresponding acid solutions as given in corresponding acid solutions as shown in Table 1. Table 1 shows three polymers that can directly be contacted with the aqueous solutions of these acids.

TABLE 1
Polymers and counter ion sources.

| | | | Polydiallyldi- |
|---|---|---|---|
| | Polyethylene- | Betainized | methylammonium |
| | imine | PEI | chloride |
| Polymer | (PEI) | (B-PEI) | (PDADMACl) |
| Counter | 1) hydrochloric | 1) HCl | 1) HCl |
| ion source | acid (HCl) | 2) acetic acid | 2) acetic acid |
| | 2) acetic acid | 3) citric acid | 3) citric acid |
| | 3) citric acid | 4) boric acid | 4) boric acid |
| | 4) boric acid | | |

As described herein, these polymeric solutions have antipathogenic properties. As all the pre-polymeric solutions are polyelectrolytes and readily soluble in distilled water, the concentration of ions in solution will be about 0.5% wt to about 5% wt. Before testing each polymer's antipathogenic properties, cytotoxicity of PEI, B-PEI and PDADMACl with different anion as shown in Table 1 are tested with common cytotoxicity test of MTT assay (see examples herein).

Provided herein are antimicrobial compositions. Such compositions are useful for the treatment of surfaces (e.g., face masks) for the reduction or prevention of transmission of microbes (e.g., bacteria, fungus, and/or viruses). In some embodiments, the composition comprises a cationic polymer selected from the group consisting of: a polydiallyldimethylammonium salt, a branched polyethyleneimine salt, polydiallyldimethylammonium with different counter ions, a chemically modified polyethyleneimine salt, or a combination thereof. The average molecular weight of branched polyethyleneimine can be 800, 1300, 2000, 10000, 25000, 60000, 75000, 270000 or 750000 g/mol.

In some embodiments, the composition further comprises a carrier. In some embodiments, the carrier comprises water, alcohol, glycerol, polyethylene glycol, or a combination thereof. In some embodiments, the carrier comprises water. In some embodiments, the carrier comprises alcohol. In some embodiments, the carrier comprises glycerol. In some embodiments, the carrier comprises polyethylene glycol.

In some embodiments, the alcohol is selected from the group consisting of ethanol, propanol, methanol, isopropyl alcohol, and combinations thereof. In some embodiments, the alcohol is selected from the group consisting of ethanol, propanol, and combinations thereof. In some embodiments, the alcohol is ethanol. In some embodiments, the alcohol is propanol. In some embodiments, the alcohol is a mixture of ethanol and propanol.

In some embodiments, the salt is a halide salt, an acetate salt, a citrate salt, a borate salt, a phosphate salt, or a combination thereof. In some embodiments, the salt is a chloride salt, a bromide salt, or an iodide salt. In some embodiments, the salt is an acetate salt, a citrate salt, a borate salt, or a phosphate salt.

In some embodiments, the halide salt is selected form the group consisting of sodium chloride, potassium chloride, potassium iodide, lithium chloride, copper(II) chloride, silver chloride, calcium chloride, chlorine fluoride, organohalides (e.g., bromomethane or isoform), hydrogen chloride, or hydrogen bromide, and combinations thereof.

In some embodiments, the acetate salt is selected from the group consisting of sodium acetate, aluminum acetate, ammonium acetate, or potassium acetate, and combinations thereof.

In some embodiments, the citrate salt is selected from the group consisting of sodium citrate, aluminum citrate, or potassium citrate, and combinations thereof.

In some embodiments, the borate salt is selected from the group consisting of sodium metaborate, borax, lithium metaborate, lithium tetraborate, zinc borate, disodium octaborate tetrahydrate, and combinations thereof.

In some embodiments, the phosphate salt is selected from the group consisting of monosodium phosphate (anhydrous), mono sodium phosphate (monohydrate), monosodium phosphate (dihydrate), disodium phosphate (anhydrous), disodium phosphate (dihydrate), disodium phosphate (heptahydrate), disodium phosphate (octahydrate), disodium phosphate (dodecahydrate), trisodium phosphate (anhydrous, hexagonal), trisodium phosphate (anhydrous, cubic), tri sodium phosphate (hemihydrate), trisodium phosphate (hexahydrate), trisodium phosphate (octahydrate), trisodium phosphate (dodecahydrate), and combinations thereof.

In some embodiments, the polydiallyldimethylammonium with different counter ions is an ion exchanged polydiallyldimethylammonium salt. In some embodiments, the chemically modified polydiallyldimethylammonium salt is a polydiallyldimethylammonium with different counter ions. In some embodiments, the chemically modified polyethyleneimine salt is a betainized polyethyleneimine salt. In some embodiments, the chemically modified polyethyleneimine salt is a sulfobetainized polyethyleneimine salt. In some embodiments, the chemically modified polyethyleneimine salt is a phosphobetainized polyethyleneimine salt.

These polymeric solutions are tested for their antipathogenic properties. As all the pre-

polymeric solutions are polyelectrolytes and readily soluble in distilled water, the concentration of solution can be about 0.1% to about 10% by weight. In some embodiments, the composition comprises from about 0.1% to about 10% by weight of the cationic polymer. In some embodiments, the composition comprises about 0.5% to about 10% by weight of the cationic polymer, about 1.0% to about 10% by weight of the cationic polymer, about 1.5% to about 10% by weight of the cationic polymer, about 2.0% to about 10% by weight of the cationic polymer, about 2.5% to about 10% by weight of the cationic polymer, about 3.0% to about 10% by weight of the cationic polymer, about 3.5% to about 10% by weight of the cationic polymer, about 4.0% to about 10% by weight of the cationic polymer, about 4.5% to about 10% by weight of the cationic polymer, about 5.0% to about 10% by weight of the cationic polymer, about 5.5% to about 10% by weight of the cationic polymer, about 6.0% to about 10% by weight of the cationic polymer, or about 6.5% to about 10% by weight of the cationic polymer.

In some embodiments, the composition comprises about 0.5%, about 1%, about 2% by weight, about 3% by weight, about 4% by weight, about 5% by weight, about 6% by weight, about 7% by weight, about 8% by weight, about 9% by weight, or about 10% by weight of the cationic polymer.

In some embodiments, the composition is about 0.1% to about 10% by weight of polydiallyldimethylammonium salt. In some embodiments, the composition is about 0.1% to about 10% by weight of polyethyleneimine salt. In some embodiments, the composition is about 0.1% to about 10% by weight of a polydiallyldimethylammonium with different counter ions. In some embodiments, the composition is about 0.1% to about 10% by weight of a chemically modified polyethyleneimine salt.

In some embodiments, the composition is about 0.5% to about 5% by weight of polydiallyldimethylammonium salt. In some embodiments, the composition is about 0.5% to about 5% by weight of polyethyleneimine salt. In some embodiments, the composition is about 0.5% to about 5% by weight of a polydiallyldimethylammonium with different counter ions. In some embodiments, the composition is about 0.5% to about 5% by weight of a chemically modified polyethyleneimine salt.

In some embodiments, the composition comprises about 0.5% by weight of polydiallyldimethylammonium salt. In some embodiments, the composition comprises about 0.5% by weight of polyethyleneimine salt. In some embodiments, the composition comprises about 0.5% by weight of a polydiallyldimethylammonium with different counter ions (e.g., HCL, acetic acid, citric acid, boric acid). In some embodiments, the composition comprises about 0.5% by weight of a chemically modified polyethyleneimine salt.

In some embodiments, the composition is about 1% to about 5% by weight of polydiallyldimethylammonium salt. In some embodiments, the composition is about 1% to about 5% by weight of polyethyleneimine salt. In some embodiments, the composition is about 1% to about 5% by weight of a polydiallyldimethylammonium with different counter ions. In some embodiments, the composition is about 1% to about 5% by weight of a chemically modified polyethyleneimine salt.

In some embodiments, the composition comprises about 1% by weight of polydiallyldimethylammonium salt. In some embodiments, the composition comprises about 1% by weight of polyethyleneimine salt. In some embodiments, the composition comprises about 1% by weight of a polydiallyldimethylammonium with different counter ions. In some embodiments, the composition comprises about 1% by weight of a chemically modified polyethyleneimine salt.

In some embodiments, the composition comprises about 2% by weight of polydiallyldimethylammonium salt. In some embodiments, the composition comprises about 2% by weight of polyethyleneimine salt. In some embodiments, the composition comprises about 2% by weight of a polydiallyldimethylammonium with different counter ions. In some embodiments, the composition comprises about 2% by weight of a chemically modified polyethyleneimine salt.

In some embodiments, the composition comprises about 3% by weight of polydiallyldimethylammonium salt. In some embodiments, the composition comprises about 3% by weight of polyethyleneimine salt. In some embodiments, the composition comprises about 3% by weight of a polydiallyldimethylammonium with different counter ions. In some embodiments, the composition comprises about 3% by weight of a chemically modified polyethyleneimine salt.

In some embodiments, the composition comprises about 4% by weight of polydiallyldimethylammonium salt. In some embodiments, the composition comprises about 4% by weight of polyethyleneimine salt. In some embodiments, the composition comprises about 4% by weight of a polydiallyldimethylammonium with different counter ions. In some embodiments, the composition comprises about 4% by weight of a chemically modified polyethyleneimine salt.

In some embodiments, the composition comprises about 5% by weight of polydiallyldimethylammonium salt. In some embodiments, the composition comprises about 5% by weight of polyethyleneimine salt. In some embodiments, the composition comprises about 5% by weight of a polydiallyldimethylammonium with different counter ions. In some embodiments, the composition comprises about 5% by weight of a chemically modified polyethyleneimine salt.

In some embodiments, the composition comprises about 6% by weight of polydiallyldimethylammonium salt. In some embodiments, the composition comprises about 6% by weight of polyethyleneimine salt. In some embodiments, the composition comprises about 6% by weight of a polydiallyldimethylammonium with different counter ions. In some embodiments, the composition comprises about 6% by weight of a chemically modified polyethyleneimine salt.

In some embodiments, the composition comprises about 7% by weight of polydiallyldimethylammonium salt. In some embodiments, the composition comprises about 7% by weight of polyethyleneimine salt. In some embodiments, the composition comprises about 7% by weight of a polydiallyldimethylammonium with different counter ions. In some embodiments, the composition comprises about 7% by weight of a chemically modified polyethyleneimine salt.

Also provided herein are processes for preparing an antibacterial, antifungal, and/or antiviral surface on a material, the method comprising treatment of at least one surface of the material with an effective amount of a composition as described herein.

Also provided herein are methods of reducing exposure of a subject to a bacterial, fungal, and/or viral infection, the method comprising applying an effective amount of a composition as described herein to at least one surface of a material worn by the subject.

In some embodiments, the material is clothing. In some embodiment, the clothing is made of a material selected from the group consisting of cotton, linen, silk, wool, polyester, or a combination thereof. In some embodiments, the material is a face mask.

In some embodiments, the material is a hard surface. In some embodiments, the material of the hard surface is selected from the group consisting of stone (e.g., granite, quartz, marble, soapstone, quartzite, etc.), concrete, glass, laminate material, metal (e.g., steel, iron, copper, etc.), wood, composite wood, plastic, and combinations thereof. In some embodiments, the hard surface comprises steel, granite, quartz, marble, plastic, concrete, or a combination thereof.

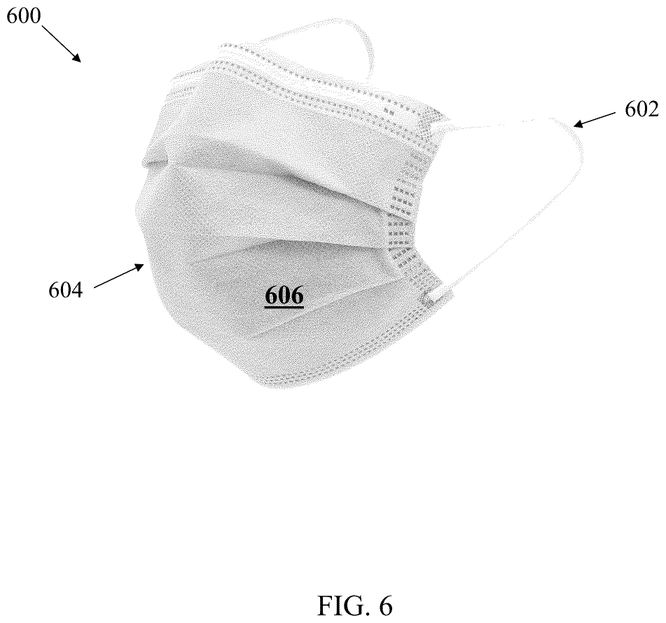

FIG. 6 depicts an example of a face mask. FIG. 6 illustrates a face mask 600 comprising a fastening member 602 and a body member 604 with an outermost layer 606. Provided herein are face masks e.g., face mask 600 comprising: a fastening member 602 for attaching a body portion 604 of the face mask to a user; and a body portion 604 joined to the fastening member 602 and configured to be placed over the mouth and at least part of the nose of the user such that inhaled air is drawn through said body portion 604, the body portion 604 comprising a plurality of layers with at least an outermost layer 606 treated with a composition as described herein.

In some embodiments, a face mask 600 comprises a fastening member 602 for attaching a body portion 604 of the face mask to a user; and a body portion 604 joined to the fastening member 602 and configured to be placed over the mouth and at least part of the nose of the user such that inhaled air is drawn through said body portion 604, the body portion comprising a plurality of layers with at least an outermost layer 606 treated with a compositions described herein.

In some embodiments, a face mask 600 comprises a body portion 604 configured to be placed over a mouth and at least part of a nose of a user such that inhaled air is drawn through said body portion 604, wherein said body portion 604 comprises an outer layer 606 treated with an effective amount of a composition described herein.

In some embodiments, the body portion 604 of the face mask 600 is treated with the composition by dipping and squeezing, spraying, ink jet printing, and combinations thereof. In some embodiments, the body portion 604 of the face mask is treated with the composition by dipping and squeezing. For example, a face mask 600 can be dipped into a composition such that the composition saturates the face mask 600. The saturated face mask can then be wrung or squeezed to remove the excess composition from the face mask 600. In some embodiments, the face mask 600 is treated with the composition by spraying the composition on at least the body portion 604 of the face mask 600. For example, any one of the compositions described herein can be mechanically or manually misted or sprayed onto at least the body portion 604 of the face mask. In some embodiments, the face mask 600 has the composition applied to at least the body portion 604 of the face mask 600 by an ink jet printing method. For example, a printer can be configured to deposit a composition onto at least the body portion 604 of the face mask 600.

In some embodiments, the face mask 600 is a PPE face mask. In some embodiments, the face mask 600 is a surgical face mask. In some embodiments, the material of the face mask 600 is selected from the group consisting of cotton, linen, silk, wool, polyester, or a combination thereof. In some embodiments, the material of the face mask 600 is a non-woven material.

Also provided herein are methods for deactivating a bacterium, fungus, and/or virus on a surface, the method comprising treating the surface with an effective amount of a composition as described herein.

In some embodiments, the composition is present in an amount of at least about 0.01% to about 25% by weight in solution when it is applied to the surface. For example, about 0.01% to about 5%, about 0.01% to about 10%, about 0.01% to about 15%, about 15% to about 20%, about 15% to about 25%, about 10% to about 25%, about 5% to about 25%, about 1% to about 25% by weight on the surface (e.g., the outermost layer 606).

In some embodiments, the composition is selected from the group consisting of polydiallyldimethylammonium salt, a polyethyleneimine salt, a polydiallyldimethylammonium with different counter ions, a chemically modified polyethyleneimine salt and combinations thereof, and is present in an amount of at least 0.01% to about 25% by weight on the surface a material.

In some embodiments, the composition is selected from the group consisting of polydiallyldimethylammonium salt, a polyethyleneimine salt, a polydiallyldimethylammonium with different counter ions, a chemically modified polyethyleneimine salt and combinations thereof, and is present in an amount of at least 0.01% to about 5% by weight on the surface a material.

In some embodiments, the composition is selected from the group consisting of polydiallyldimethylammonium salt, a polyethyleneimine salt, a polydiallyldimethylammonium with different counter ions, a chemically modified polyethyleneimine salt and combinations thereof, and is present in an amount of at least 0.01% to about 10% by weight on the surface a material.

In some embodiments, the composition is selected from the group consisting of polydiallyldimethylammonium salt, a polyethyleneimine salt, a polydiallyldimethylammonium with different counter ions, a chemically modified polyethyleneimine salt and combinations thereof, and is present in an amount of at least 0.01% to about 15% by weight on the surface a material.

In some embodiments, the composition is selected from the group consisting of polydiallyldimethylammonium salt, a polyethyleneimine salt, a polydiallyldimethylammonium with different counter ions, a chemically modified polyethyleneimine salt and combinations thereof, and is present in an amount of at least 15% to about 25% by weight on the surface a material.

In some embodiments, the composition is selected from the group consisting of polydiallyldimethylammonium salt, a polyethyleneimine salt, a polydiallyldimethylammonium with different counter ions, a chemically modified polyethyleneimine salt and combinations thereof, and is present in an amount of at least 20% to about 25% by weight on the surface a material.

In some embodiments, the compounds and compositions described herein can be used to treat microbial infections and disease. In some embodiments, the compounds described herein can treat viral, bacterial, and fungal infections in a subject in need thereof (e.g., diagnosed or identified as having a viral, fungal, or bacterial infection).

In some embodiments, the composition can be used as a broad-spectrum antimicrobial agent to treat infections in animals. In some embodiments, the animal is human. In some embodiments, the animal is an animal being treated in a veterinary setting. In some embodiments, the composition is in a solution suitable for application for administration to an eye.

In some embodiments, the composition is a solution to treat eye infections in animals. In some embodiments, the solution is applied as a gel or ointment to skin infections. In some embodiments, the solution is applied prior to surgery and in the postoperative period as a gel or ointment to be applied as a prophylaxis against broad spectrum antimicrobial infection.

In some embodiments, a method of treating a viral infection in a subject in need thereof, comprises administering to the subject a therapeutically effective amount of a polydiallyldimethylammonium salt, a branched polyethyleneimine salt, a chemically modified polydiallyldimethylammonium salt, a chemically modified polyethyleneimine salt, or a combination thereof. In some embodiments, the method of treating a viral infection in a subject in need thereof, comprises administering to the subject a composition comprising a therapeutically effective amount of a polydiallyldimethylammonium salt, a branched polyethyleneimine salt, a chemically modified polydiallyldimethylammonium salt, a chemically modified polyethyleneimine salt, or a combination thereof; and a pharmaceutically acceptable carrier.

In some embodiments, a method of treating a fungal infection in a subject in need thereof, comprising administering a therapeutically effective amount of a polydiallyldimethylammonium salt, a branched polyethyleneimine salt, a chemically modified polydiallyldimethylammonium salt, a chemically modified polyethyleneimine salt, or a combination thereof. In some embodiments, the method of treating a fungal infection in a subject in need thereof, comprises administering to the subject a composition comprising a therapeutically effective amount of a polydiallyldimethylammonium salt, a branched polyethyleneimine salt, a chemically modified polydiallyldimethylammonium salt, a chemically modified polyethyleneimine salt, or a combination thereof; and a pharmaceutically acceptable carrier or a combination thereof. In some embodiments, the fungus that causes the fungal infection is from the Candida genus. In some embodiments, the fungus that causes the fungal infection is Candidia albicans.

In some embodiments, a method of treating a bacterial infection in a subject in need thereof, comprising administering a therapeutically effective amount of a polydiallyldimethylammonium salt, a branched polyethyleneimine salt, a chemically modified polydiallyldimethylammonium salt, a chemically modified polyethyleneimine salt, or a combination thereof. In some embodiments, the method of treating a bacterial infection in a subject in need thereof, comprises administering to the subject a composition comprising a therapeutically effective amount of a polydiallyldimethylammonium salt, a branched polyethyleneimine salt, a chemically modified polydiallyldimethylammonium salt, a chemically modified polyethyleneimine salt, or a combination thereof; and a pharmaceutically acceptable carrier or a combination thereof. In some embodiments, the fungus that causes the fungal infection is from the Escherichia, Bacillus, Staphylococcus, Pseudomonas, and combinations thereof. In some embodiments, the bacteria that causes the bacterial infection is E. coli, S. aureus, P. aeruginosa, and B. subtilis or combinations thereof.

In some embodiments, the term ‘about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “carrier” refers to an agent that is useful in preparing antimicrobial compositions. The carrier can be a single compound or a blend of compounds that are used in amounts effective to solubilize and/or disperse the ingredients of the antimicrobial composition. For example, carriers can include solvents such as water and alcohol.

As used herein, the phrase “chemically modified” refers to the reaction of one or more primary amines on a polymer as provided herein with a reactive agent to prepare one or more secondary amines. In some embodiments, the resulting secondary amine comprises a moiety having a negative charge. In some embodiments, the primary amine is chemically modified with a betaine (e.g., a sulfobetaine, a carboxybetaine, or a phosphobetaine) to prepare a betainized polymer (i.e., the polymer has been betainized).

As used herein, the term “salt” refers to salts that retain the desired activity of the subject compound and exhibit minimal undesired effects. These salts may be prepared during the final isolation and purification of the compound, or by separately reacting the purified compound in its free acid or free base form with a suitable base or acid, respectively. In some embodiments, salts may be preferred over the respective free base or free acid because such salts impart greater stability or solubility to the molecule thereby facilitating formulation. Basic compounds are generally capable of forming acid addition salts by treatment with a suitable acid. Suitable acids include inorganic acids and organic acids. Representative acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (to sylate),napthalene-2-sulfonate, ethanedisulfonate, and 2,5-dihydroxybenzoate.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents, and buffers), for preventing the action of microorganisms (e.g., antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

As used herein, the term “administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal, or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal, or mucosal route of administration, for example, intranasally, vaginally, rectally, sublingually, or topically. Administration can also be performed, for example, once, a plurality of times, and/or over one or more extended periods.

The term “modulate”, “modulator”, and/or “modulating” as used herein refers to modification of chemical and/or biological activity. The modification can include inhibition of chemical and/or biological activity, controlling an influence of chemical and/or biological activity, and/or an activation of biological and/or chemical activity.

The phrase “therapeutically effective amount” refers to the administration of an amount of a polydiallyldimethylammonium salt, a branched polyethyleneimine salt, a chemically modified polydiallyldimethylammonium salt, a chemically modified polyethyleneimine salt, or a combination thereof to modulate microbial load relative to an untreated subject.

The term “inhibiting”, “inhibitor”, and/or “inhibits”, as used herein refers to ceasing biological and/or chemical activity, slowing biological and/or chemical activity, and/or reducing biological and/or chemical activity.

As used herein the term “antimicrobial” refers to an agent that can inhibit or eliminate a microbe. Such microbes can include microbes from one of the two prokaryotic domains, Bacteria and Archaea, as well as microbes such as viruses, fungi, and protists.

As used herein, the phrases an “effective amount” of an antimicrobial composition refers to an amount of the composition sufficient enough to reduce or eliminate one or more microbes. Effective amounts of an antimicrobial composition will vary with the kind of antimicrobial agent chosen, the particular surface or surfaces being treated with the antimicrobial composition, the specific components of the composition being used, and like factors.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, database entries, and other references mentioned herein are incorporated by reference in their entirety. In case of conflict, the present specification, including definitions, will control.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: the Betainization of PEI

PEI is a branched cationic polymer that is well-known for its highly effective gene delivery capacity and antibacterial properties. However, the toxic nature of PEI has restricted its use in widespread biomedical applications. Chemical modification e.g., betainization of PEI (see FIG. 4) makes this material biocompatible and useful for the purposes herein. Betainization of PEI: 20 ml of PEI solution was treated with 10 ml of 0.2 M NaOH solution at 500 rpm and precipitated in excess amounts of acetone. Upon decanting acetone and drying, PEI solution in water and 1,3-propane solution in water at 1:1.5 mole ratio of PEI reacted 12 hours at room temperature leading to betainized PEI (B-PEI). Then, 2 wt % B-PEI aqueous solutions can be prepared as a spray used in the experiments described herein. This made the product biocompatible. Using a commercially available spray bottle, the compound was successfully sprayed and the compound was bound onto cotton masks and surgical masks.

### Example 2: Antibacterial and Antifungal Capabilities of PEI-Based Polymers

Anti-bacterial and anti-fungal features of PEI-HCl, PEI-BA, PEI-CA, and B-PEI were studied on E. coli, B. subtilis, S. aureus and P. aeruginosa bacteria and C. albicans fungal strains as model organisms using known Broth-Macro Dilution method. Minimum inhibitory concentration (MIC) of the samples were determined based on the lowest sample concentration that prevented visible microbial growth. Minimum bactericidal or fungicidal concentration (MBC or MFC) was assessed on the basis of lowest sample concentration that has achieved 99% killing activity of microorganisms. More simply, this refers to the lowest sample concentration that yielded no visible growth in both main culture and accompanying subcultures in the agar plates. MIC MBC and MFC of the samples against specified microorganisms were illustrated in Table 2.

TABLE 2
MIC, MBC, and MFC values of PEI-based polymers against E. coli (gram−), S. aureus (gram+),
B. subtilis (gram+) and P. aeruginosa (gram−), bacteria and C. albicans fungal strains.

| | E. coli | | P. aeruginosa | | C. Albicans | | S. aureus | | B. Subtilis | |
|---|---|---|---|---|---|---|---|---|---|---|
| | (gram−) | | (gram−) | | (fungi) | | (gram+) | | (gram+) | |
| | MIC | MBC | MIC | MBC | MIC | MFC | MIC | MBC | MIC | MBC |
| Polymers | (mg/mL) | (mg/mL) | (mg/mL) | (mg/mL) | (mg/mL) | (mg/mL) | (mg/mL) | (mg/mL) | (mg/mL) | (mg/mL) |
| PEI-HCl | 2.5 | 10 | 2.5 | 5 | 2.5 | 5 | 2.5 | 10 | 2.5 | 5 |
| PEI-BA | 2.5 | 10 | 5 | 10 | 5 | 10 | 2.5 | 5 | 2.5 | 10 |
| PEI-CA | 5 | 20 | 10 | 20 | 10 | 20 | 10 | 20 | 5 | 20 |
| B-PEI | 5 | 20 | 5 | 20 | 10 | 20 | 5 | 20 | 5 | 20 |

MIC values of gentamycin antibiotic for C. albicans fungal strain and E. coli, B. subtilis, S. aureus for P. aeruginosa bacteria were reported to be 0.0025 mg/mL, 0.008 mg/mL, 0.004 mg/mL, 0.01 mg/mL, and 0.004 mg/mL, respectively.

As shown in Table 2, the lowest MIC, MBC, and MFC values were obtained for PEI-HCl polymers with 2.5-5 mg/mL MIC values for all microorganisms. The MBC and MFC values of PEI-HCl were determined to be 5 mg/mL for all organisms except E. coli bacteria which is 10 mg/mL. PEI-BA polymers attained second highest anti-microbial activities with 2.5 mg/mL MIC concentration for E. coli (gram −), S. aureus (gram +), and B. subtilis (gram +) strains whereas 5 mg/mL MIC values were observed for P. aeruginosa (gram −) bacterial, and C. albicans fungal strains. The third material having lowest MIC values was B-PEI polymers, which appeared to be 5 mg/mL for all bacterial strains, and 10 mg/mL for C. albicans. The MIC values for PEI/CA was revealed to be 5 mg/mL for E. coli (gram−), and B. subtilis (gram+) strains while it was 10 mg/mL for P. aeruginosa, S. aureus, and C. Albicans.

The MBC and MFC of PEI-CA, and B-PEI shared same values of 20 mg/mL concentration whereas those of the PEI-BA were found to be 10 mg/mL for all organisms except for S. aureus which was realized to be 5 mg/mL. In a general and collective prospect of the antimicrobial performances of the PEI based polymers, PEI-HCl and PEI-/BA polymers are the most salient ones having orderly the highest anti-bacterial and anti-fungal performances. The most efficient antimicrobial activity of PEI/HCl may be ascribable to strong acidity of HCl, which may result in the best protonation amongst others. Moreover, native BA has inherent antimicrobial properties when combined with PEI. While reasonable antimicrobial effects might have coalesced in PEI-BA polymers, it was not as much as the activity of PEI-HCl. Additionally, B-PEI formed by betainization of PEI also demonstrated shrinking antimicrobial activities that are about half of PEI-HCl in terms of MIC values and at least one fourth by MBC and MFC of PEI-HCl. On the other hand, PEI-CA polymers had milder yet similar antimicrobial performances against the tested strains.

### Antiviral Capabilities of PEI Based Materials

Antiviral properties of PEI based polymer with different counter ions were tested against SARS-CoV-2 virus. For that purpose, SARS-CoV-2 virus stocks were prepared by growing virus in Vero 76 cells. PEI based solutions were prepared at 1% (by weight) in distilled water. SARS-CoV-2 virus stock was added to triplicate tubes of each prepared concentration so that there was 10% virus solution by volume and 90% prepared sample. Only media was added to one tube of each prepared concentration to serve as toxicity controls. Ethanol was tested in parallel as a positive control and distilled water serves as the virus control. The prepared compound solutions at 1% (weight/volume in DI water) and virus were incubated at room temperature for 30 minutes. Following the contact period, the solutions were neutralized by a 1/10 dilution in test media.

Surviving virus was quantified by standard end-point dilution assay. Neutralized samples were combined for quantification for the average of triplicate tests. Samples were serially diluted using eight 10-fold dilutions in test medium. Each dilution was added to 4 wells of a 96-well plate with 80-100% confluent Vero E6 cells. The toxicity controls were added to an additional 4 wells and 2 of these wells were infected with virus to serve as neutralization controls, ensuring that residual sample in the titer assay plated did not inhibit growth and detection of surviving virus. Plates were incubated at 37±2° C. with 5% CO₂. On day 5, post-infection plates were scored for presence or absence of viral cytopathic effect (CPE). The Reed-Muench method was used to determine end-point titers (50% cell culture infectious dose, CCID50) of the samples, and the log reduction value (LRV) of the compound compared to the negative (water) control was calculated. Virus controls were tested in DI water and the reduction of virus in test wells compared to virus controls was calculated as the log reduction value (LRV). Toxicity controls were tested with media not containing virus to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus (approximately 30 CCID50) that would produce an observable amount of CPE during the incubation period.

Virus titer and log reduction value (LRV) for samples tested against SARS-CoV-2 are shown in Table 3.

TABLE 3
The antiviral activities of PEI based compounds against SARS-
CoV-2 at 30 min incubation at 22 ± 2° C.

| | | Contact | | Neut. | | | |
|---|---|---|---|---|---|---|---|
| | Concen- | Time | Toxic- | Con- | Virus | VC | |
| Compound | tration | (min) | ity^{a} | trol^{b} | Titer^{c} | Titer^{c} | LRV^{d} |
| DI Water | 100% | 30 | None | None | 4.3 | 4.3 | 0 |
| PEI | 1% | 30 | 1/100 | None | 3.3 | 4.3 | 1.0 |
| PEI-HCl | 1% | 30 | 1/100 | None | <2.7 | 4.3 | >1.6 |
| PEI-BA | 1% | 30 | 1/100 | None | 4.3 | 4.3 | 0 |
| PEI-CA | 1% | 30 | 1/100 | None | <2.7 | 4.3 | >1.6 |
| Ethanol | 70% | 30 | None | None | <0.7 | 4.3 | >3.6 |

^{a}Cytotoxicity indicates the highest dilution of the endpoint titer where full (80-100%) cytotoxicity was observed.
^{b}Neutralization control indicates the highest dilution of the endpoint titer where compound inhibited virus CPE in wells after neutralization (ignored for calculation of virus titer and LRV).
^{c}Virus titer of test sample or virus control (VC) in log10 CCID50 of virus per 0.1 mL.
^{d}LRV (log reduction value) is the reduction of virus in test sample compared to the virus control.

As shown in Table 3, the tested compounds exhibit antiviral activity. As shown in the table, bare (unmodified) PEI can reduce virus 90% (LRV 1.0). On the other hand, PEI-HCl (HCl treated/modified PEI) and PEI-CA (Citric acid treated/modified PEI) can reduce virus >90% (LRV>1.6) at the studied concentration, 1%.

The virus control titer, DI water, was 4.3 log CCID50 per 0.1 mL and was used for comparison of all test sample titers to determine LRV. Samples with <1 log reduction are not considered active for virucidal activity.

The limit of detection of virus for samples that did not exhibit cytotoxicity when plated for endpoint dilution assay was 0.7 log CCID50 per 0.1 mL. When >80% cytotoxicity was observed in wells of diluted samples, presence of virus could not be ruled out and therefore the limit of detection was altered. For instance, when cytotoxicity was seen in the 1/10 dilution the limit of detection was 1.7 logs, in 1/100 it was 2.7 logs, and so forth.

As can be seen from Table 4, the betainized forms of PEI (B-PEI, B-PEI-HCl, B-PEI-BA) exhibited virucidal activity, reducing SARS-CoV-2 titer by more than 3 log CCID50 per 0.1 mL (>99.9%).

TABLE 4
B-PEI based compounds that show virucidal activity against SARS-
CoV-2 after 30 min incubation at 22 ± 2° C.

| | | Contact | | Neut. | | | |
|---|---|---|---|---|---|---|---|
| | Concen- | Time | Toxic- | Con- | Virus | VC | |
| Compound | tration | (min) | ity^{a} | trol^{b} | Titer^{c} | Titer^{c} | LRV^{d} |
| B-PEI | 1% | 30 | None | None | 0.7 | 4.3 | 3.6 |
| B-PEI-HCl | 1% | 30 | None | None | <0.7 | 4.3 | >3.6 |
| B-PEI-BA | 1% | 30 | None | None | <0.7 | 4.3 | >3.6 |
| B-PEI-CA | 1% | 30 | None | None | 4.0 | 4.3 | 0.3 |

^{a}Cytotoxicity indicates the highest dilution of the endpoint titer where full (80-100%) cytotoxicity was observed.
^{b}Neutralization control indicates the highest dilution of the endpoint titer where compound. inhibited virus CPE in wells after neutralization (ignored for calculation of virus titer and LRV).
^{c}Virus titer of test sample or virus control (VC) in log10 CCID50 of virus per 0.1 mL.
^{d}LRV (log reduction value) is the reduction of virus in test sample compared to the virus control.

### Example 3: Cytotoxicity Tests Against L929 Fibroblasts Cells

Cytotoxicity tests against L929 fibroblasts cells were completed. Cytotoxicity of PEI and B-PEI based solutions was performed by employing MTT colorimetric assay to assess the viability of the L929 fibroblasts cell in the presence of PEI based solutions. Human L929 fibroblast cells were cultured in DMEM supplemented with 10% (v/v) FBS and 1% antibiotics as a culture medium at 37° C., with 5% CO₂. In brief, 100 μL of 5×10⁴ cell/mL concentration of the cell suspension in culture medium was seeded onto each well in a 96-well plate and incubated for 24 h at 37° C., with 5% CO₂ to obtain adhesive L929 cells. Then, the culture medium was replaced with 100 μL of different concentrations of PEI based solutions in the range of 50-1000 μg/mL in the cultured medium was added to the adhesive cells. As a control group, the culture medium was replaced with the fresh culture medium to obtain untreated cells. The plate was incubated for 24 h at 37° C., with 5% CO₂. At the end of the incubation, the PEI based solutions were removed from the wells and the cells were washed with PBS at one time. Separately, 5 mg/mL concentration of MTT reagent was diluted tenfold with DMEM and 100 μL of this reagent was put into each well. The plate was incubated for 2 hours in a dark condition and MTT solution was replaced with 200 μL of DMSO to dissolve the formazan crystals. Then, the absorbance value of the observed purple color was read by using a plate reader (Thermo, Multiskan Sky) at 570 nm wavelength. The cell viability % in the presence of the PEI solutions was calculated by the means of absorbance of the treated cells divided by the absorbance values of untreated cells (as a control) and multiplying this ratio by 100. All assays were performed three times, and the results were given with standard deviations. The statistical analysis was performed using GraphPad Prism 8 software and the differences between the groups were assessed according to the Student's t-test. The results were determined as statistically significant for the P value was *p<0.05 and **p<0.001 vs control.

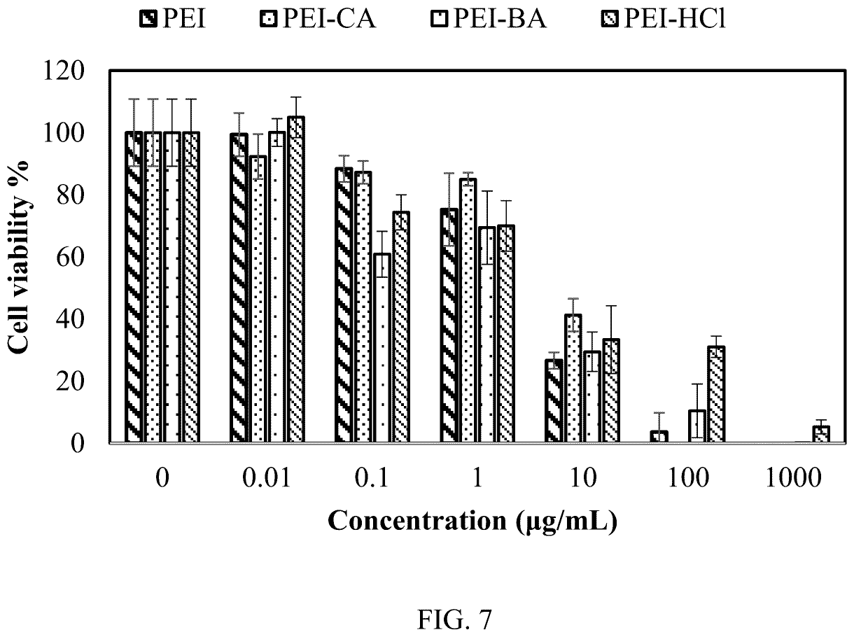

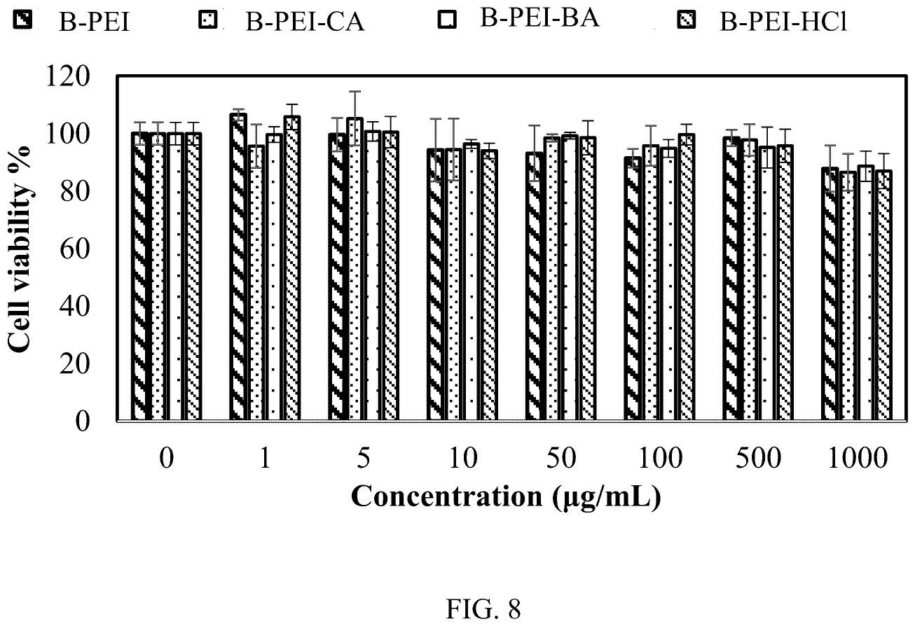

As shown in FIG. 7, the compounds PEI, PEI-CA, PEI-BA and PEI-HCl were toxic to L929 fibroblasts cell in a concentration dependent manner, e.g., all these solutions at 10 μg/mL concentration render about less than 40% cell viability and are completely toxic at concentrations >10 μg/mL, e.g., 100 and 1000 μg/mL concentration. On the other hand, as illustrated in FIG. 8, the betainized forms of PEI spray solutions are nontoxic even at 1000 μg/mL, rendering about 95% cell viabilities for all forms of B-PEI with different counter ions, e.g., B-PEI, B-PEI-CA, B-PEI-B A and B-PEI-HCl.

What is claimed is:

1. A broad-spectrum antimicrobial composition comprising:
a cationic polymer selected from the group consisting of a polydiallyldimethylammonium salt, a branched polyethyleneimine salt, a chemically modified polydiallyldimethylammonium salt, a chemically modified polyethyleneimine salt, and combinations thereof; and
a carrier.

2. The composition of claim 1, wherein the salt is a halide salt, an acetate salt, a citrate salt, a borate salt, a phosphate salt, or combinations thereof.

3. The composition of claim 1, wherein the chemically modified polydiallyldimethylammonium salt is a polydiallyldimethylammonium having a different counter ion.

4. The composition of claim 3, wherein the different counter ion may be obtained from hydrochloric acid, acetic acid, citric acid, boric acid, or combinations thereof.

5. The composition of claim 1, wherein the chemically modified polyethyleneimine salt is a betainized polyethyleneimine salt.

6. The composition of claim 5, wherein the chemically modified polyethyleneimine salt is a sulfobetainized polyethyleneimine salt or a phosphobetainized polyethyleneimine salt.

7. The composition of claim 1, wherein the composition comprises from about 0.5% to about 5% by weight of the cationic polymer.

8. A method for inhibiting antimicrobial activity on a surface of a material comprising applying an effective amount of the composition of claim 1 to the surface of the material to treat the surface of the material to inhibit the antimicrobial activity.

9. The method of claim 8, wherein the material is a fabric material, a face mask, or a hard material.

10. The method of claim 9, wherein the fabric material is configured to cover a mouth and/or at least a portion of a nose of a subject.

11. The method of claim 9, wherein the face mask comprises:
a fastening member for attaching a body portion of the face mask to a user; and
the body portion joined to the fastening member and configured to be placed over a mouth and at least part of a nose of the user such that inhaled air is drawn through the body portion, the body portion comprising a plurality of layers with at least one layer treated with the composition of claim 1.

12. The method of claim 11, wherein the composition of claim 1 is applied to the at least one layer of the face mask by a method selected from the group consisting of dipping and squeezing, spraying, ink jet printing, brushing, soaking, and combinations thereof.

13. The method of claim 9, wherein the hard material comprises steel, granite, quartz, plastic, concrete, and combinations thereof.

14. The method of claim 8, wherein the composition is present on the surface in an amount of at least about 0.01% to about 20% by weight.

15. A method of treating a microbial infection in a subject in need thereof, comprising:
administering a therapeutically effective amount of a polydiallyldimethylammonium salt, a branched polyethyleneimine salt, a chemically modified polydiallyldimethylammonium salt, a chemically modified polyethyleneimine salt, and combinations thereof; and
a pharmaceutically acceptable carrier.

16. The method of claim 15, wherein the microbial infection is a bacterial infection, a fungal infection, a viral infection, or combinations thereof.

17. The method of claim 16, wherein the bacterial infection is caused by a bacteria selected from the genus Escherichia, Bacillus, Staphylococcus, Pseudomonas, and combinations thereof.

18. The method of claim 17, wherein the bacterial infection is caused by a bacteria selected from the group consisting of E. coli, S. aureus, P. aeruginosa, and B. subtilis, and combinations thereof.

19. The method of claim 16, wherein the viral infection is caused by severe acute respiratory syndrome coronavirus 2 (SARS-CoV-2).

20. The method of claim 16, wherein the fungal infection is caused by a fungus from theCandida genus.
